(19) United States
(12) Patent Application Publication
(10) Pub. No.: US 2020/0368069 A1
(43) Pub. Date: Nov. 26, 2020

(54) EAR CLEANING DEVICE

(71) Applicant: Ronald K. Horowitz, Hampton Bays, NY (US)

(72) Inventor: Ronald K. Horowitz, Hampton Bays, NY (US)

(21) Appl. No.: 16/418,550

(22) Filed: May 21, 2019

(51) Int. Cl. A61F 11/00; A61F 13/38

(52) U.S. Cl. CPC A61F 11/006; A61M 35/006; A61F 13/38

(57) ABSTRACT

A swab that may be used as an ear cleaning device including an elongate stem with an oil absorbing sheet covering a tip core at each end extending outwardly, wherein the tip core may be comprised of a soft, resilient material such as cotton. oil absorbing sheet wrapped around the tip core to form a covered swab end. The covered swab ends render the tips more oil absorbent than a typical cotton swab.

### BACKGROUND

The present disclosure relates to swabs useful in cleaning the ear or applying cosmetics.

Swabs having an absorbent material on the tip and an elongated stem are well known. Cotton is generally used as the absorbent tip material. Stem materials are often comprised of wood, rolled paper or plastic. Conventional swabs are typically constructed by applying an absorbent material directly to the distal ends of the stem. An adhesive may be used to more firmly hold the absorbent material in place on the swab. In order to properly mount the swab tip on the stick, a number of prior art patents have generally disclosed placing a thin adhesive coating on the stick at the point at which the swab tip is to be placed. Examples of patents which carry such a disclosure include the following: U.S. Pat. Nos. 2,705,009; 3,179,108; 3,255,494; 3,443,562; 3,586,380; and 4,259,955.

While conventional swabs can be effective in removing earwax, the cotton material typically used in the swab is not ideal for absorbing oil. While cotton does have some oil absorbing capability, cotton is used in a swab because it is soft, resilient and unlikely to cause damage to human skin or tissue. A cotton tip is easily inserted into an ear. The cotton may grip and remove wax mechanically, however, other materials are known to be more effective in absorbing and removing oil.

Oil absorbing sheets comprised of paper are known to be used to remove facial oil. For example, natural or synthetic papers using vegetable fibers, synthetic pulp or kenaf have been used in oil absorbing sheets. To improve smoothness, these papers may be calendered and/or coated with powders such as calcium carbonate and sizing agents to create a smooth oil absorbing sheet.

Improvements to oil absorbing papers are described in Japanese Kokai No. 4-45591, which teaches adhering porous spherical beads onto the surface of an oil absorbing paper so as to solve the problems caused by calendering or coating of paper with powders such as calcium carbonate powders. These beads also are used to allegedly increase the capacity of the papers to absorb sebum. Japanese Unexamined Patent Publication (Kokai) No. 6-319664 discloses a high-density oil absorbing paper produced by mixing (a) a pulp material containing vegetable fibers, as the main component with (b) an inorganic filler, followed by paper-making to form a paper with a basis weight of 0.7 g/cm2 or more.

An oil absorbing paper for sebum is also disclosed in Japanese Examined Patent Publication (Kokoku) No. 56-8606, or U.S. Pat. No. 4,643,939, which describes a cosmetic oil absorbing paper produced by mixing hemp fibers with 10 to 70% by weight of polyolefin resin fibers and making a paper with a basis weight of from 12 to 50 g/cm2. Japanese Unexamined Utility Model Publication (Kokai) No. 5-18392, discloses an oil absorbing synthetic paper comprising an oil absorbing paper with a smooth surface coating of inorganic or organic powder material such as clay particles, silica fine-particles, and powdered fibers.

Japanese Unexamined Patent Publication (Kokai) No. 9-335451 (WO99/29220) discloses an oil wipe made of a porous thermoplastic film. This oil absorbing wipe film has higher oil absorption capacity than the oil absorbing papers and is also superior in confirming removal of oil following wiping as compared to oil absorbing papers.

European Patent No. 1,066,826 discloses a skin cleansing sheet with an oil absorbing layer. Although porous thermoplastic films are disclosed as possible oil absorbing layers, the preferred oil absorbents sheets may be comprised of nonwoven lipophilic fiber fabrics which contain a powder such as silica.

It is evident that further improvements are necessary in swab technology for the purpose of cleaning an ear. These improvements should focus on introducing materials with better oil absorbing properties than cotton at the end of a swab.

Accordingly, it is an object of the present invention to provide a swab with better oil absorption than those currently available.

Another object of the present invention is to provide a swab with an oil absorbing properties than cotton.

Yet another object of the present disclosure is to maintain the size and softness of a conventional cotton swab while improving oil absorbing properties of the swab.

### SUMMARY

The present disclosure relates to an ear cleaning device, an improvement on a cotton swab, including an elongate stem with an oil absorbing sheet covering a tip core at each end extending outwardly wherein the tip core may be comprised of a soft fabric with an oil absorbing sheet wrapped around the central portion to form a covered swab end. The covered swab ends render the tips more oil absorbent than a typical cotton swab that may be used for ear cleaning or other cleaning purposes.

### BRIEF DESCRIPTION OF THE DRAWINGS

FIG. 1 shows a front view of a conventional swab;

FIG. 2 shows a front view of an adhesive oil absorbing sheet;

FIG. 3A shows a front view of an adhesive oil absorbing sheet being wrapped around a swab end; FIG. 3B shows a front view of an adhesive oil absorbing sheet fully wrapped around a cotton swab end and adhered to the stem;

FIG. 4 shows a front view of a non-adhesive oil absorbing sheet;

FIG. 5A shows a front view of a non-adhesive oil absorbing sheet being wrapped around a cotton swab end; FIG. 5B shows a front view of an elastic band according to the present disclosure; FIG. 5C shows a front view of a non-adhesive oil absorbing sheet wrapped around a cotton swab end and attached with an elastic band;

FIG. 6A shows a rear view of an elastic absorbing sheet according to the present dislosure; FIG. 6B shows an elastic absorbing sheet attached to a cotton swab end and adhered to the stem.

### DETAILED DESCRIPTION

The present disclosure relates to swab that may be used as an ear cleaning device. The swab is a personal hygiene device for removing wax from an ear, such as the removal of ear wax from the ear canal of a user's ear. It should be understood that, as in the case of a cotton swab, although the primary use of the ear cleaning device is for cleaning the ear, the ear cleaning device may be used for the removal of any bodily secretions, waste or debris, such as mucus, from any orifice of the body that can receive the tips of the device. It should be understood that the ear cleaning device may be used for any desired purpose and is not only provided for the cleaning of a user's ears; the ear cleaning device is multi-functional, similar to the variety of functions for which one would use a conventional cotton swab.

According to the present disclosure, ear cleaning device includes an elongate stem with a covered tip core at each end extending outwardly and having a tip core comprised of a soft fabric with an oil absorbing sheet wrapped around the tip core. The covered tip core renders the tips more oil absorbent than a typical cotton swab that may be used for ear cleaning or other cleaning purposes.

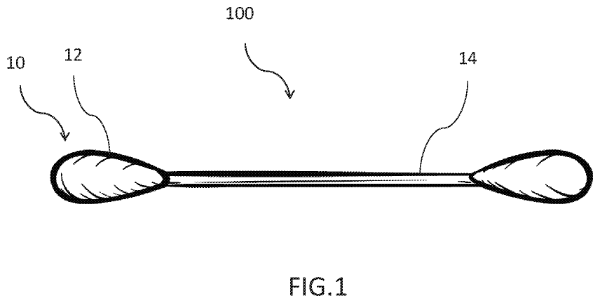

With regard to FIG. 1, a swab 100 according to the prior art is shown. Components of swab 100 include an elongate stem 14 at each end extending outwardly and having a tip core 10 comprised of a support material 12, which in one embodiment may be a soft fabric such as cotton.

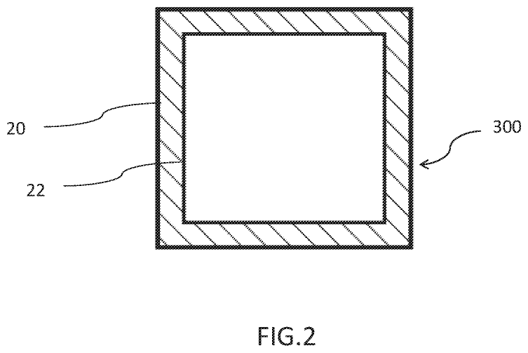

With regard to FIG. 2, an oil absorbing sheet 300 is shown. Oil absorbing sheet 300 may be comprised of a sheet of fibrous material coated with an oil absorbing substance. Oil absorbing sheet 300 may be comprised of a thin porous paper which may be coated with an oil absorbing substance. Oil absorbing sheet 300 may, in some embodiments of the present disclosure, may be comprised of polyester, polypropylene, wood pulp, or rayon fibers, thermoplastic fibrous material, cellulosic fibrous papers. In one embodiment, oil absorbing sheet may be approximately the thickness of conventional cigarette rolling paper, which may in one embodiment have a basis weight of between 10 grams per square meter (gsm) and 100 gsm. In other embodiments, oil absorbing sheet 300 may be thicker. In one embodiment, oil absorbing sheet 300 may be comprised of hemp fibers with 10 to 70% by weight of polyolefin resin fibers and making a paper with a basis weight of from 10 gsm to 100 gsm. In one embodiment, oil absorbing sheet 300 may be comprised of pulp material containing vegetable fibers, as the main component, with an inorganic filler, followed by paper-making to form a paper with a basis weight of between 10 and 100 gsm or more. In one embodiment, oil absorbing sheet 300 may be comprised of an open celled hydrophobic polypropylene film. In one embodiment, oil absorbing sheet 300 may be comprised of a porous hydrophobic film. In one embodiment, oil absorbing sheet 300 may be comprised of a polymer sheet of hydrophobic polypropylene. In one embodiment, oil absorbing sheet 300 may be comprised of a cosmetic tissue comprised of a mixture of transparent synthetic resin fibers with nontransparent oil absorbing plant fibers. In one embodiment, oil absorbing sheet 300 may be comprised of a porous polytetrafluoroethylene membrane layered over a flexible backing. In one embodiment, oil absorbing sheet 300 may be comprised of an open-celled, microporous and hydrophobic polymeric film.

In one embodiment, oil absorbing sheet 300 may be comprised of bleached kraft pulp consisting mainly of cellulose and hemicellulose, two polymers that are composed of sugar molecules, wherein glucose units are linked together (beta-1-4) such that they are resistant to chemical hydrolysis. Kraft fibers are highly porous. The most important group of pores are slit-like and have a width between about 5 and 20 nm. Native cellulose fibers have a central void area called a lumen. Kraft fibers usually have a small amount of residual lignin and extractives.

To generate bleached kraft pulp, market pulps are usually received as bales having a moisture content in the range of 10 to 20%, and these are dispersed with strong agitation in a hydropulper tank. Most grades of paper require the pulp to be refined by passing a slurry of 2 to 5% solids content between rotating metal bars of a refiner. The refining can be optimized by controlling the pH to the weakly alkaline range, controlling the energy content per mass of fibers and controlling the freeness of the resulting pulp.

Oil absorbing sheet 300 may be coated with an oil absorbing or otherwise oil removing substance. Coating may be carried out using any conventional means such as nozzle spraying, dipping, or saturating with an oil absorbing substance and coating may be carried out before or after affixing the oil absorbing sheet 300 to the tip core 10. In one embodiment, oil absorbing sheet 300 may be may be coated with inorganic or organic powder material such as clay particles, silica fine-particles, and powdered fibers. In one embodiment, oil absorbing sheet 300 may be coated with adhering porous spherical beads. In one embodiment, oil absorbing sheet 300 may be saturated with astringents. Astringents are known for their ability to remove excess oil. Astringents may include, but are not limited to, hammelis virginiana (witch hazel), potassium alum (Alum) or other generally non-toxic alum. Other astringents may be utilized according to the present disclosure, as would be known to one of ordinary skill in the art, where such astringents may include alum, acacia, sage, yarrow, witch hazel, bayberry, distilled vinegar, very cold water, and rubbing alcohol. Astringent preparations may include silver nitrate, potassium permanganate, zinc oxide, zinc sulfate, Burow's solution, tincture of benzoin, and such vegetable substances as tannic and gallic acids.

Oil absorbing sheet 300 may, in one embodiment be coated with a composition know for treating a blockage caused by ear wax, or ceruminosis. Ear wax removing compositions may include, but are not limited to, carbamide peroxide in a 6.5% solution, wherein the oil absorbing sheet 300 is saturated with carbamide peroxide solution. Other cerumenolytic chemicals used for softening the cerumen and lubricating the canal, and include sodium bicarbonate, hydrogen peroxide, acetic acid, glycerin, acetone, triethanolamine polypeptide, various forms of docusate sodium and a combination of aluminum acetate, dichlorobenzene and benzethonium chloride.

Oil absorbing sheet 300 may be coated with surfactants such as sodium cocoate, sodium riciniolate, sodium tallow and the like, as would be known to one of ordinary skill in the art. Oil absorbing sheet 300 may further include emollients, preservatives, coating agents, penetrants skin conditioners.

In one embodiment, oil absorbing sheet 300 is comprised of bleached kraft pulp treated with polyaminedeamine epichlorhydrin and coated with potassium alum, or aluminum sulfate, and combined with polyacrylamide.

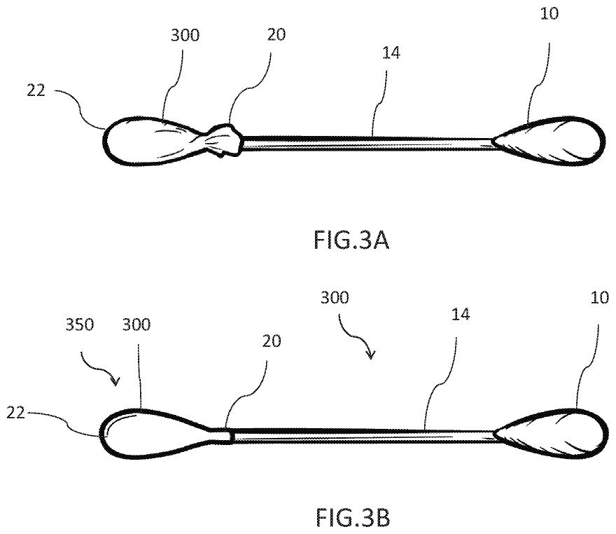

With regard to FIG. 3A, with an adhesive oil absorbing sheet 300 may be wrapped around tip core 10, which is comprised of support material 12, which may be, in one embodiment, comprised of cotton, as would be known to one of ordinary skill in the art. Adhesive portion 20, at the edges of adhesive oil absorbing sheet 300, may be coated with an adhesive, including sealants such as heat sealants. Adhesives may include, but are not limited to a wax, animal glues, plant glues, synthetic monomer glues, synthetic polymer glues as would be known to one of ordinary skill in the art. In one embodiment, an adhesive may be applied directly to stem 14 to affix oil absorbing sheet 300. The adhesive may be dried after affixing oil absorbing sheet 300 to stem 14 prior to use of the ear cleaning device. Oil absorbing sheet 300 will remain flush with stem 14 after applying the adhesive and during which period of time the adhesive will dry. The adhesive may be dried under at least one of heat and pressure.

In one embodiment, adhesive portion 20 is coated with a wax applied to the perimeter of oil absorbing sheet 300. Bunch wrapping may be used, as is well known in the art of wrapping candies such as lollipops, and disclosed in the background of WO2004071892 and U.S. Pat. No. 1,370,980. A bunchwrap package, where the confectionery (candy), or herein tip core 10, is placed in the middle of a generally square and flat piece of packaging material, herein oil absorbing sheet 300, and where the four corners of oil absorbing sheet, which may be of other shapes than square, are folded towards stem 14 and wrapped around it by a twisting motion. This version of packaging is represented in FIG. 1A of the '892 application. Historically, packaging materials (substrates) used for these lollipop wrappers are mainly wax-coated paper, and regenerated cellulose film (cellophane), although, alternatively, adhesive portion may be coated with a heat-sealable lacquer. Heat sealable materials which may also herein be referred to as adhesives or sealers include, but are not limited to, wax, a heat sealable lacquer, and other heat sealable materials as would be known to one of ordinary skill in the art. A certain amount of heat is applied to the film in order to heat-seal it into its twisted position depending on the thickness of the material and the adhesive, as would be known to one of ordinary skill in the art.

Given that a certain temperature on the inside of the wrapping material must be reached in order to seal it, it is generally known, by those skilled in the art of packaging, a higher temperature must be set on the heat source (e.g. sealing jaws) which contacts the outside of the wrapping material. Sealing jaws may be a hinged and form a hollow cylinder comprised of metal, wherein the hollow portion is shaped and sized to match stem 14. When the two halves of the sealing jaws are heated and closed to enclose stem 14 and the wrapped adhesive portion 20 of oil absorbing sheet 300 forms a seal is formed and adhesive portion 20 of oil absorbing sheet 300 is sealed flush with stem 14 adjacent tip core 10. The temperature difference needed to seal adhesive portion 20 to stem 14 is adjustable and increases with increasing material thickness and with decreasing seal contact time, as would be known to one of ordinary skill in the art.

The adhesive may be applied on at least one of or preferably both of an inner side and an outer side at the edges of adhesive oil absorbing sheet 300 such that when wrapped around stem 14, adhesive oil absorbing sheet 300 adheres to stem 14, thereby forming a covering that matches the bulbous shape of tip core 10 to form covered swab end 350 as shown in FIG. 3B. Wrapping and sealing may in some embodiments be performed by hand or by machine. Wrapping would generally involve either clockwise or counterclockwise rotation of the perimeter of oil absorbing sheet 300 around stem 14 until the perimeter surface of oil absorbing sheet 300 is flush with stem 14 and oil absorbing sheet 300 conforms to the bulbous shape of tip core 10. Covered swab end 350 renders the tips more oil absorbent than a typical cotton swab, while retaining the advantages of a conventional cotton swab for ear cleaning purposes.

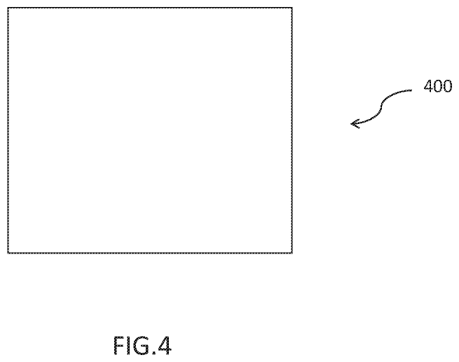

With regard to FIG. 4, another embodiment of the present disclosure is shown. FIG. 4 shows a non-adhesive oil absorbing sheet 400. Non-adhesive oil absorbing sheet 400 may be comprised of materials included in the present disclosure to comprise adhesive oil absorbing sheet 300, without adhesive portion 20.

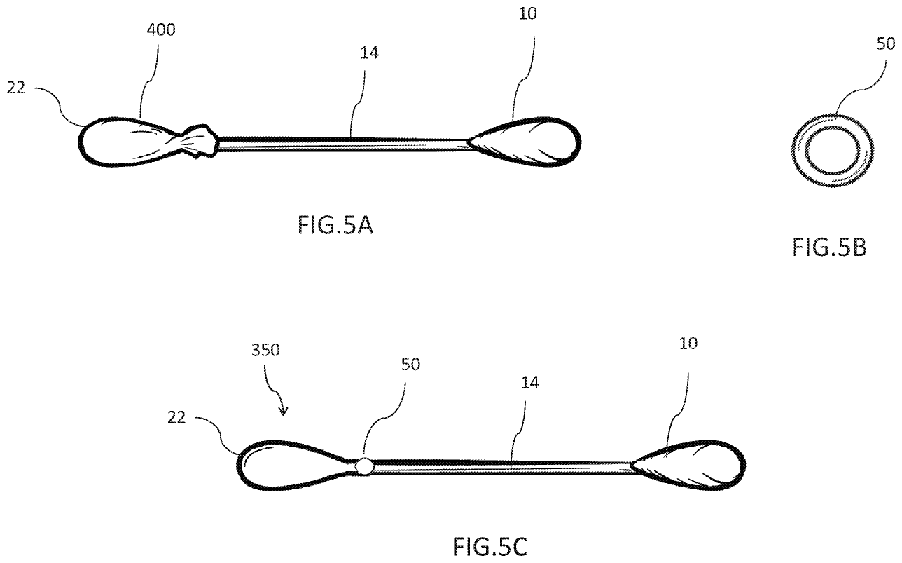

FIG. 5A shows non-adhesive oil absorbing sheet 400 being wrapped around tip core 10. Once wrapped around tip core 10, non-adhesive oil absorbing sheet 400 may be, in one embodiment, secured to stem 14 with an elastic band 50. Elastic band 50 is shown in FIG. 5B. Elastic band 50 may be comprised of elastomers in the class of polymer materials with high elastic nature including natural rubber, synthetic rubber, nitrile rubber, silicone rubber, urethane rubbers, chloroprene rubber, Ethylene Vinyl Acetate (EVA rubber), synthetic elastic materials and the like, as would be known to one of ordinary skill in the art. Once elastic band 50 is secured to non-adhesive oil absorbing sheet 400 on stem 14, covered swab end 350 is formed, as shown in FIG. 5C.

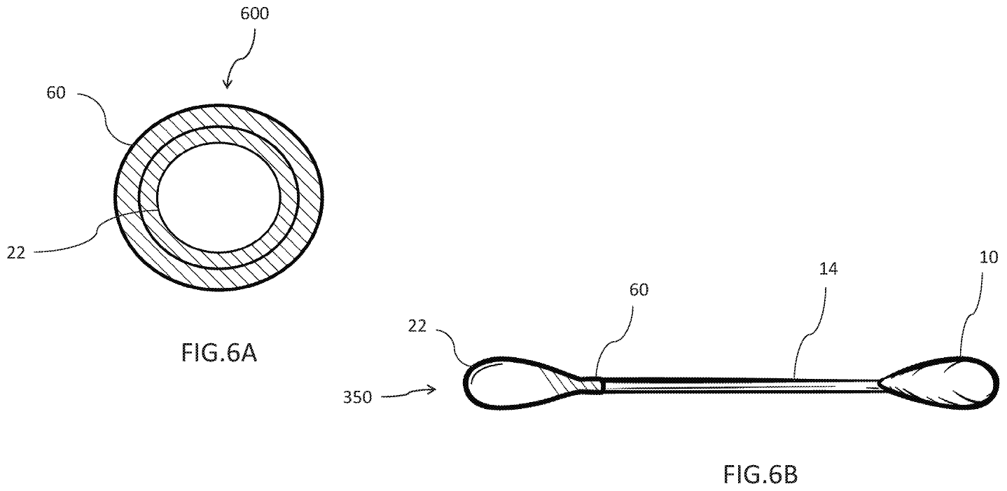

With regard to FIGS. 6A and 6B, an elastic oil absorbing sheet 600 is shown. Elastic oil absorbing sheet 600 may be comprised of an elastic portion 60 and a terminal portion 22. Elastic portion 60, which is comprised of an elastic material may be affixed or adhered to central portion 22 which is comprised of an oil absorbing sheet material. Elastic portion 60 may be adhered using conventional adhesives as described herein and as would be known to one of ordinary skill in the art. Elastic portion 60 may be comprised of any elastic material as would be known to one of ordinary skill in the art, natural rubber, synthetic rubber, nitrile rubber, silicone rubber, urethane rubbers, chloroprene rubber, Ethylene Vinyl Acetate (EVA rubber), synthetic elastic materials and the like, as would be known to one of ordinary skill in the art. Elastic portion 60 may cover approximately 50% of tip core 10, while the range may include approximately 40% to greater than 50% of the length of tip core 10 and be located on a side proximal to stem 14. Elastic portion 60 of oil absorbing sheet 600 may be stretched to fit over tip core 10 and stem 14.

It is to be understood that the drawings and descriptive matter are in all cases to be interpreted as merely illustrative of the principles of the invention, rather than as limiting the same in any way, since it is contemplated that various changes may be made in various elements to achieve like results without departing from the spirit of the invention or the scope of the appended claims. While the drawings shown herein depict an oil absorbing sheet covering a tip core on only one end, both ends may be covered according to the present disclosure. All documents cited in the Detailed Description of the invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

What is claimed is:

1. A swab comprising:
a stem having an elongate structure with a first end and a second end;
the stem being adhered at a stem end to a tip core having a bulbous shape comprised of a support material, wherein the support material is comprised of a soft, resilient material;
an oil absorbing sheet covering the tip core and affixed to the stem adjacent the tip core such that the oil absorbing sheet generally matches the bulbous shape of the tip core to form a covered swab end;
a coating on the oil absorbing sheet, the coating comprised of at least one oil removing substance.

2. The swab of claim 1, wherein the oil absorbing sheet is coated with at least one of an astringent and a surfactant.

3. The swab of claim 2, wherein the astringent is potassium alum.

4. The swab of claim 1, wherein the oil absorbing sheet is comprised of a bleached kraft pulp coated with potassium alum.

5. The swab of claim 1, wherein the oil absorbing sheet has at least one adhesive portion located at a perimeter of the oil absorbing sheet.

6. The swab of claim 5, wherein the oil absorbing sheet has a first adhesive portion on an outer portion of the oil absorbing sheet and a second adhesive portion on an inner portion of the oil absorbing sheet, such that the oil absorbing sheet can be wrapped around and adhered to the stem to form a surface flush with the stem.

7. The swab of claim 6, wherein the first adhesive portion and the second adhesive portion are coated with a heat sealable material and heat sealed to the stem to create a flush surface with the stem.

8. The swab of claim 7, wherein the heat sealable material is a wax.

9. The swab of claim 1, wherein the oil absorbing sheet has an elastic portion.

10. The swab of claim 9, wherein the elastic portion is comprised of at least one of a natural rubber and a synthetic rubber.

11. The swab of claim 9, wherein the elastic portion covers at least 40% of a length of the tip core; wherein the elastic portion is proximal to the stem.

12. The swab of claim 1, wherein the oil absorbing sheet includes a ceruminolytic agent.

13. The swab of claim 1, wherein the oil absorbing sheet has a basis weight of between 10 gsm and 200 gsm.

14. The swab of claim 13, wherein the oil absorbing sheet is comprised of a fibrous material.

15. A method for manufacturing an ear cleaning device, comprising:
adhering a support material to an end of a stem to form a tip core, wherein the tip core is generally bulbous in shape;
an oil absorbing sheet covering the tip core and affixed to the stem adjacent the tip core such that the oil absorbing sheet generally matches the bulbous shape of the tip core to form a covered swab end;
applying an adhesive to at least one of a first adhesive portion on an outer portion of the oil absorbing sheet and a second adhesive portion on an inner portion of the oil absorbing sheet to form an adhesive portion;
covering the tip core with the oil absorbing sheet;
wrapping a perimeter of the oil absorbing sheet around the stem in either a clockwise or counterclockwise direction;
sealing the adhesive portion of the oil absorbing sheet to the stem adjacent the tip core.

16. The method of claim 15, further comprising coating the oil absorbing sheet with at least one of an astringent and a surfactant.

17. The method of claim 15, further comprising selecting an oil absorbing sheet from a fibrous material, where a basis weight of the oil absorbing sheet is between 10 and 100 gsm.

18. A swab comprising:
a stem having an elongate structure with a first end and a second end;
the stem being adhered to a tip core comprised of a support material adhered at a stem end, wherein the support material is comprised of cotton;
an oil absorbing sheet covering the tip core such that the oil absorbing sheet generally matches a bulbous shape of the tip core to form a covered swab end;
wherein the oil absorbing sheet has an adhesive portion comprised of an inner surface adhesive portion located at a perimeter of the oil absorbing sheet and an outer surface adhesive portion located at a perimeter of the oil absorbing sheet;
wherein the adhesive portion is wrapped around and sealed to the stem to form a surface flush with the stem,
a coating on the oil absorbing sheet, the coating comprised of at least one oil removing substance;
wherein the oil absorbing sheet contains at least one of potassium alum and a surfactant;
wherein the oil absorbing sheet is comprised of bleached kraft pulp;
wherein the oil absorbing sheet has a basis weight of between 10 gsm and 100 gsm.
